(19) United States
(12) Patent Application Publication
(10) Pub. No.: US 2022/0265443 A1
(43) Pub. Date: Aug. 25, 2022

(54) PERIPHERAL BRAIN-MACHINE INTERFACE SYSTEM VIA VOLITIONAL CONTROL OF INDIVIDUAL MOTOR UNITS

(71) Applicant: The Regents of the University of California, Oakland, CA (US)

(72) Inventors: Jose M. Carmena, Berkeley, CA (US); Emanuele Formento, Berkeley, CA (US); Paul Abraham Botros, Berkeley, CA (US); Michel M. Maharbiz, Berkeley, CA (US)

(21) Appl. No.: 17/691,994

(22) Filed: Mar. 10, 2022

Related U.S. Application Data
(63) Continuation of application No. PCT/US2020/052529, filed on Sep. 24, 2020.
(60) Provisional application No. 62/906,516, filed on Sep. 26, 2019.

(51) Int. Cl. A61F 2/72; A61B 5/389; A61B 5/375; A61B 5/256; A61N 1/36; G06F 3/01

(52) U.S. Cl. CPC A61F 2/72; A61B 5/389; A61B 5/375; A61B 5/256; A61N 1/36003; G06F 3/015; A61B 2562/0219; A61H 2201/5007

(57) ABSTRACT

A brain-machine interface (BMI) system includes one or more implantable or non-implantable sensors, each being configured to detect or measure electrophysiological activity of motor units and to transmit an electrophysiological activity signal; one or more wearable apparatuses configured to be worn by or attached to a user and configured to receive and process the one or more electrophysiological activity signals transmitted by the sensors, and configured to transmit the processed signals to one or more processing units, which are configured to produce control signals based on the received processed signals using one or more machine learning algorithms; and one or more effectors configured to receive the control signals and configured to transduce the control signals into a haptic, tactile, chemical, mechanical, auditory, visual, and/or electrical stimuli so as to provide feedback to a user and/or to control operation of an external effector.

### CROSS REFERENCES TO RELATED APPLICATIONS

This Patent Application is a continuation of PCT Application No. PCT/US2020/052529 by Jose M. Carmena et al., entitled “PERIPHERAL BRAIN-MACHINE INTERFACE SYSTEM VIA VOLITIONAL CONTROL OF INDIVIDUAL MOTOR UNITS,” filed Sep. 24, 2020, which claims priority to U.S. Provisional Patent Application No. 62/906,516 by Jose M. Carmena et al., entitled “PERIPHERAL BRAIN-MACHINE INTERFACE SYSTEM VIA VOLITIONAL CONTROL OF INDIVIDUAL MOTOR UNITS,” filed Sep. 26, 2019, each of which is incorporated herein by reference in its entirety.

### BACKGROUND

Brain-machine interfaces (BMIs) create an artificial link between intentions and actions that bypasses the musculoskeletal system. Brain activity is captured with neural interfaces and translated into control signals using decoding algorithms. This technology has the potential to revolutionize the way people interact with each other and with the external environment, for example, allowing people with severe paralysis to regain independence and empowering the average consumer with a direct connection to the digital world. In the clinical domain, proof-of-concept studies have already demonstrated remarkable results, with paraplegic subjects using BMI to control robotic arms, computer cursors, or even their own paralyzed limb through electrical stimulation. However, to effectively extract control signals from our brain, current BMIs require highly invasive neural interfaces that present significant associated risks. Indeed, the implantation procedure involves brain surgery, during which a piece of skull is removed, electrodes are lowered into the brain, and a connector—used to wire the neural interface to external recording devices—is mounted on the skull. For those with a debilitating injury or disease, such as tetraplegia or stroke, the relative risk of electrode implantation and maintenance might be worth the benefit of a BMI, but this is not true for many, and especially not for the average consumer. Non-invasive brain recording technologies exist, such as the electroencephalogram (EEG), but they either lack the temporal or spatial resolution necessary for effectively powering a BMI.

An alternative to detect intentions from the brain is to target the nervous system at the muscle level. Motor unit activity can be detected using surface, epimysial, and intramuscular electromyography (EMG): surface EMG has the advantage of being non-invasive but suffers from some limitations (e.g., movement artifacts, crosstalk, and poor recordings stability); epimysial and intramuscular EMG largely overcome these limitations but are more invasive. Few systems have exploited this technology to extract control signals from motor commands and operate prostheses, orthoses, or consumer devices. For example, in transradial amputees, surface EMG signals recorded from the forearm muscles are sometimes used to detect intended hand movements and control a prosthetic hand. The number of functions that these systems can control is limited by the number of functions controlled by the targeted muscles. Since different motor units from the same motor pool are recruited in a fixed order, a maximum of one function can be controlled from one muscle (in practice, multiple muscles are controlled in synergy with others and can be hardly controlled independently). This bandwidth might be enough for effectively controlling prostheses or arbitrary effectors with a limited number of degrees of freedom, but it is insufficient when more degrees of freedom are necessary or when only a few muscles can be controlled by the user (as in the case of tetraplegic people or subjects with large amputations).

### SUMMARY

The present disclosure provides BMI systems that combine minimally invasive or non-invasive motor unit recordings with neurofeedback, e.g., to extract more than one degree of freedom per targeted muscle.

The various embodiments leverage the ability of people to learn to control individual motor units independently of one another when provided with a sensory feedback signal linked to these unit potentials. This type of abstract skill learning capitalizes on the native neural circuitry for motor learning and therefore has great potential to feel naturalistic, generalize well to novel movements and environments, and benefit from the nervous system's highly-developed storage and retrieval mechanisms for skilled behavior.

In an embodiment, a brain-machine interface (BMI) system is provided that includes one or more implantable or non-implantable sensors, each of the one or more sensors being configured to detect or measure electrophysiological activity of motor units (each motor unit comprising a motor neuron and the skeletal muscle fibers innervated by that motor neuron's axonal terminals) and to transmit an electrophysiological activity signal; one or more wearable apparatuses configured to be worn by or attached to a user and configured to receive and process the one or more electrophysiological activity signals transmitted by the one or more sensors, and configured to transmit the processed signals to one or more processing units; the one or more processing units, configured to receive the processed signals from the one or more wearable apparatuses and to produce control signals based on the received processed signals using one or more machine learning algorithms; and one or more effectors configured to receive the control signals and configured to transduce the control signals into a haptic, tactile, chemical, mechanical, auditory, visual, and/or electrical stimuli so as to provide feedback to a user and/or to control operation of an external effector.

According to certain embodiments, the one or more wearable apparatuses process the one or more electrophysiological activity signals by applying one or more of a filtering algorithm, a down-sampling algorithm, a signal detection algorithm, and additional mathematical transformations to the one or more electrophysiological activity signals.

According to certain embodiments, at least one of the one or more implantable sensors includes one or multiple electrodes and an RF transceiver.

According to certain embodiments, at least one of the one or more sensors is non-invasive and positioned on the skin near targeted nerves or muscles.

According to certain embodiments, at least one of the one or more sensors is includes a non-invasive high-density grid of surface EMG electrodes.

According to certain embodiments, the high-density grid includes a grid of electrodes with a minimum of 16 electrodes and a maximum inter-electrode distance of 10 mm.

According to certain embodiments, the one or multiple electrodes are configured to be implanted intradermally, intramuscularly or on the epimysium of a targeted muscle.

According to certain embodiments, the one or multiple electrodes are configured to be implanted on the epineurium or within the nerve innervating a targeted muscle.

According to certain embodiments, the one or more effectors include at least one neurofeedback effector.

According to certain embodiments, the one or more effectors include at least one external effector.

According to certain embodiments, the at least one external effector comprises one of a computing device, a mechanical actuator, a mechanical transducer, an exoskeleton, a robotic manipulandum, an exoskeleton, a prosthesis, or a smart phone.

According to an embodiment, a non-transitory computer-readable medium is provided that stores instructions, which when executed by one or more processors cause the one or more processors to: receive one or more processed signals from one or more wearable apparatuses, each of the one or more processed signals representing measured electrophysiological activity of a motor unit of a user; produce control signals based on the received processed signals using one or more statistical models and/or trained machine learning algorithms; and transmit the control signals to one or more effectors configured to transduce the control signals into a haptic, tactile, chemical, mechanical, auditory, visual, and/or electrical stimuli so as to provide feedback to the user and/or to control operation of an external effector. Each wearable apparatus may be communicably coupled to, or integrated with, one or more sensors as described herein. In an embodiment, the one or more effectors include at least one external effector, and wherein the at least one external effector comprises one of a computing device, an exoskeleton, a prosthesis, or a smart phone

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF DRAWINGS

FIG. 1 illustrates a BMI system according to an embodiment.

FIG. 2 illustrates a calibration mode, according to an embodiment, which personalizes the motor unit detection and selection model to the user, increasing reliability of control of the BMI.

FIG. 3 illustrates a practice mode, according to an embodiment, which allows users to train their ability to control the BMI.

FIG. 4 illustrates a control mode, according to an embodiment, wherein external effector signals, Z, are enabled.

FIG. 5a shows a schematic representation of the operant conditioning experiment used to train rats to control individual motor units of a targeted muscle: a 16-channel intramuscular electrode array is used to capture neuromuscular electrophysiology signals from the targeted muscle; motor unit activity is extracted using a spike sorting algorithm (previously trained on baseline data) and used to control an auditory signal that is fed back to the rat; rats are rewarded when producing a target tone that requires them to generate a specific pattern of motor unit activity; patterns of activity leading to reward are adapted by the controller module to test rats' ability to control different motor units independently.

FIG. 5b illustrates evidence of independent control of individual motor units: raw neuromuscular activity recorded from the implanted electrode, during two conditioning paradigms (#1, left; #2, right), in one rat; in the first paradigm the subject was trained to activate a specific ensemble of motor units (E1: ensemble of units detected from electrode channel 10) while suppressing a second ensemble (E2: ensemble of units recorded from all channels with the exception of channel 10); in the second paradigm, the two ensembles were switched; highlighted boxes show the waveforms of the detected action potentials generated by the reinforced motor units (E1).

FIG. 5c illustrates dimensionality of neuromuscular data: bar plots representing the explained variance of the first, second, and the remaining (3+) principal components of the firing rates of the detected motor units, during the last three sessions of each of the two conditioning paradigms (error bars indicate the standard deviation).

FIG. 6 shows a block diagram of an implementation of a peripheral brain-machine interface embodiment in healthy human subjects using non-invasive neuromuscular sensors: boxes indicate the software and hardware components, while arrows indicate data streams involved in the three different operating modes of the implemented BMI: (1) calibration, (2) exploration, and (3) training and exploitation; neuromuscular signals are measured using a high-density grid of surface EMG electrodes from the biceps muscle; during the calibration mode, the detection model used to extract motor unit activity is initialized using the measured neuromuscular signals; during the exploration mode, the controller module transforms detected motor unit activity into auditory and visual neurofeedback signals while the subject attempts strategies to activate different motor units independently—in this mode, both the detection model and the controller are continuously updated, respectively, to improve detection performance and to select as source of control the motor units that shows the strongest signs of independence; the training-and-exploitation mode is enabled once at least three motor units are deemed potentially controllable by the controller adaptation algorithm; once in training-and-exploitation mode, model and controller parameters are fixed and the controller is used to transform the activity of the three most controllable motor units into control signals for a computer mouse.

FIG. 7 illustrates volitional and selective control of three individual motor units of the biceps muscle: representative traces showing the activity measured from three bipolar channels during periods of time where a subject was able to selectively recruit three different motor units; highlighted traces indicate the neuromuscular activity that was classified as belonging to one of the recruited motor units; boxes on the right shows the median of the detected motor unit action potentials on the three different channels over a 20-minute recording.

FIG. 8a illustrates a schematic of the transformation used to convert the activity of three motor unit into 2D cursor coordinates: cursor coordinates were computed by performing the vectorial sum of three control signals with directions dividing the 2D space into three equal subspaces (i.e., with a 120 degrees angle between each other) and amplitude proportional to the firing activity of an assigned motor unit; the origin of the control signals space was set to the center of the screen.

FIG. 8b illustrates a center-out cursor task: plots shows fastest cursors trajectories during a 20-minutes center-out cursor task where subjects were instructed to reach with the cursor different targets appearing on the screen; targets on the first row required selective activation of a single motor unit; targets on the second row required the simultaneous activation of two different motor units with equal amplitude; text annotation indicate the target number and the shortest time required by the subject to reach each target.

FIG. 8c illustrates control signal space: heat maps showing pairwise bivariate distributions of the three extracted control signals; each observation represents a 16 ms window.

### DETAILED DESCRIPTION

The following detailed description is exemplary in nature and is not intended to limit the invention or the application and uses of the invention. Furthermore, there is no intention to be bound by any expressed or implied theory presented in the following detailed description or the appended drawings.

According to various embodiments, a brain-machine interface (BMI) system is provided that can record the activity of individual motor units of one or multiple muscles, provide a form of biofeedback linked to the recorded activity to the user, and transform this activity into control signals that can be transmitted to, and acted upon by, external devices.

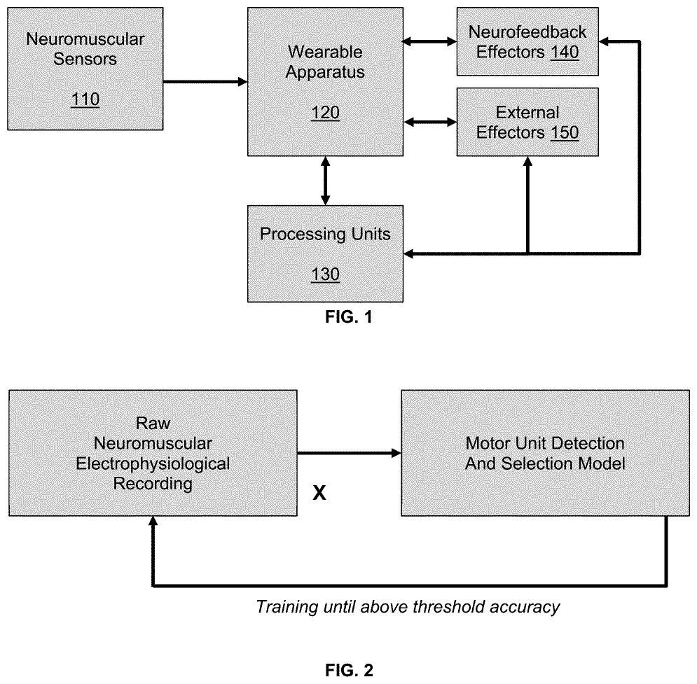

FIG. 1 illustrates a BMI system 100 according to an embodiment. BMI system 100 includes one or multiple implantable or non-implantable sensors 110, one or multiple wearable apparatuses 120, one or multiple processing units 130, one or multiple neurofeedback effectors 140, and/or one or more external effectors 150. In an embodiment, the one or multiple sensors 110 may be implanted in a minimally invasive procedure to detect the electrophysiological activity of motor units and transmit the captured signals, e.g., to the wearable apparatus 120 and/or directly to the processing unit(s) 130. In another embodiment, the one or multiple sensors 110 may be contained within the wearable apparatus and positioned on the surface of the skin. The one or multiple wearable apparatuses 120 receive and process the signals transmitted by the sensors, and transmit the processed signals to the processing unit(s) 130. The processing unit(s) 130 may each include an external processing unit or an embedded processing unit, or both an embedded processing unit and an external processing unit. Each processing unit may itself comprise one or more processors and associated circuitry and memory. The one or multiple processing units 130 operate to receive and process sensor signals and manage statistical models and/or execute trained machine learning algorithms to reliably and accurately produce control signals. The one or multiple neurofeedback effectors 140 transduce incoming control signals into a haptic, tactile, chemical, mechanical, auditory, visual, and/or electrical stimuli. The one or more external effectors 150 receive and act on the control signals produced by the one or more processing units 130 in any of various ways, depending on the type of external effector used.

In one embodiment, an implantable sensor component 110 may use microwire electrodes for electrophysiological sensing and radio frequency (RF) coupling for wireless power and communication. In this embodiment, the implantable sensor 110 is composed of an RF transceiver unit, implanted in a subcutaneous pocket, with multiple electrodes wired to the transceiver unit. In all embodiments, electrodes can be implanted either intradermally, intramuscularly or on the epimysium of the targeted muscles, on the epineurium or within the nerve innervating the targeted muscles, or positioned on the skin near the targeted muscles. The active sites of an electrode may be dimensioned to record the activity of one or more motor units, for example, with the active surface area of each electrode ranging between about 100 um² to about 10 mm².

In an embodiment, a wearable apparatus 120 embeds: a transceiver, used to communicate with the sensor(s) 110; a microcontroller that handles wireless communication, performs basic processing (such as filtering, down-sampling, and signal detection) on the acquired data, and may store limited amounts of data; and a bidirectional communication link to the processing unit(s) 130. The wearable apparatus 120 serves to feed data from the sensor(s) 110 to the processing unit(s) 130. In an embodiment, the sensor(s) 110 may be integrated with a wearable apparatus 120.

The processing unit(s) 130, which may be co-located within the wearable apparatus and/or reachable via network communication, govern the computational models that translate electrophysiological data into effector commands. In an embodiment, a motor unit detection and selection model transforms in real-time a neuromuscular data signal, X, into an n-dimensional signal, Y, representing neural activity. In one embodiment, neural data is transformed into population firing rates of the multi-unit activity detected via thresholds on each electrode. In this case, n is equal to the number of electrodes. In another embodiment, single-unit activity from each electrode is extracted using spike sorting algorithms and their firing rate is used to build Y. In this case, n corresponds to the number of extracted single units. In another embodiment, an estimate of the neural drive from descending inputs to the targeted muscles is computed from the aggregate motor unit activity across all electrodes. In this case, n corresponds to the estimated dimensionality of this neural drive. A mathematical transform, executed in the controller, is then used to convert a signal Y into an m-dimensional signal K or Z (with m less than or equal to n; m and n being integers) used to control effectors.

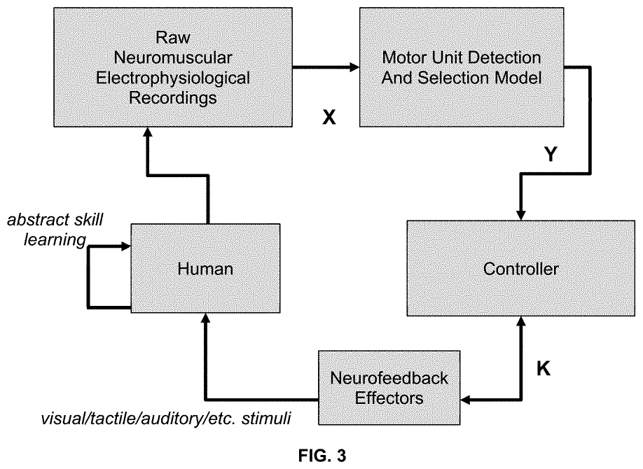

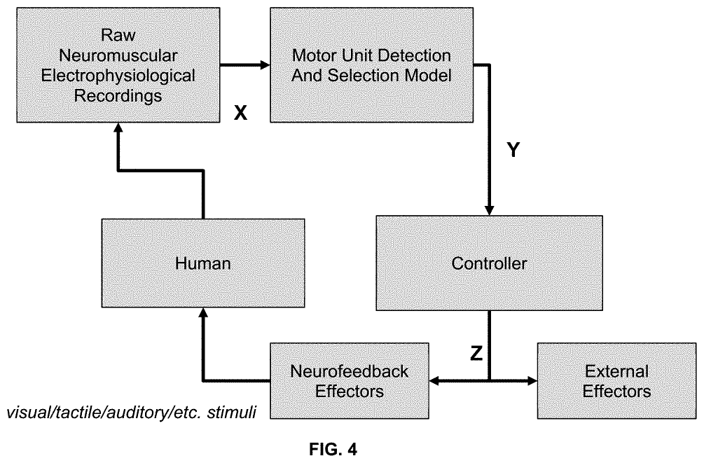

In an embodiment, one or multiple neurofeedback effectors 140 and/or one or more external effectors 150 receive control signals from the processing unit(s) 130. Neurofeedback effector(s) 140 are controlled by signals, K, to produce haptic, tactile, chemical, mechanical, auditory, visual, and/or electrical stimuli that instruct the user as to the output of the processing units (see FIG. 3). These signals may be used as feedback in order for a user to learn how to effectively operate the BMI. External effector(s) are controlled by signals, Z (see, FIG. 4). An external effector 150, such as an external computer, an exoskeleton, a prosthesis, mobile phone, or other device, receives the control signals from the processing unit(s) 130 via a predefined application programming interface (API) and can act on these signals in whichever manner is needed.

In an embodiment, the system has three operating modes: calibration, practice, and control. FIG. 2 illustrates a calibration mode, according to an embodiment, which personalizes the motor unit detection and selection model to the user, increasing reliability of control of the BMI. During this mode, the user may be instructed to perform a set of contractions in the targeted muscles, thereby generating a sufficient breadth of data measured by the sensors 110. Then, in order to allow the user to more easily and effectively control the effectors, the motor unit detection and selection model is updated to select motor units that are deemed “sufficiently easy” to independently control by the user. In one embodiment, the model selects motor units based on their order of recruitment: activity of units that are recruited with the weakest voluntary contractions are considered easier to control with respect to units that are recruited during stronger contractions. The calibration mode may continue until satisfactory performance is achieved.

FIG. 3 illustrates a practice mode, according to an embodiment, which allows users to train their ability to control the BMI. In this mode, only neurofeedback effector signals, K, are enabled; external effector signals, Z, are disabled. The user can incorporate these neurofeedback signals as a feedback signal to associate brain activity with individual motor unit activity; just as proprioception provides a feedback for muscle activity during motor skill learning, these neurofeedback signals provide feedback for individual motor unit activity to facilitate abstract skill learning. The dimensionality of K is selected by the user depending on their ability in controlling the BMI: new users should start with a number of dimensions equal to the number of targeted muscles and progressively increase the number of dimensions as they become more skilled. In one embodiment, a performance measure is used to automatically increase the number of dimensions that the subject should practice on depending on their skill level. The practice mode may continue until satisfactory proficiency is achieved.

FIG. 4 illustrates a control mode, according to an embodiment, wherein external effector signals, Z, are enabled. Using a mathematical transform, the controller maps motor unit activity signals, Y, into effector-specific control signals, Z. The dimensionality of Z depends on the number of control signals requested from the external effector. In addition to being used to control external effectors, in this modality, Z can also be used to control the neurofeedback effectors. The system is expected to be operated in the control mode for the majority of time.

The embodiments utilizing implantable sensors 110 advantageously provide a novel system based on stable, chronic, minimally invasive electrophysiological recordings and neurofeedback to volitionally produce reliable, high-throughput control signals. Existing systems do not integrate neurofeedback in their solution. In addition, most existing systems utilize neuromuscular recordings taken from the skin (“surface EMG”), which are prone to noise and cannot measure the same motor units over an extended time period. Use of stable, invasive recordings advantageously enables the various embodiments to build accurate computational models personalized to the particular user, which together with neurofeedback provide a stable platform that enables abstract skill learning.

Unlike existing non-invasive systems, embodiments utilizing non-implantable sensors exploit neurofeedback to volitionally produce reliable, high-throughput control signals. While invasive embodiments can provide better performance, non-invasive embodiments do not require implantation procedures and can be an acceptable tradeoff for some users.

The present embodiments have applications in both the medical and consumer domains. One embodiment can be used to control robotic prosthetics. In this embodiment, the targeted muscles might correspond to the residual muscles controlling the amputated limb. For example, in the case of transradial amputees, a hand prosthesis might be controlled using the residual extrinsic muscles of the hand. As compared to myoelectric control methods typically used for hand prostheses that rely on surface electrophysiological recordings, the present embodiment leverages its stably implanted electrodes for both higher throughput and higher accuracy. Similarly, another example of use could be to power an exoskeleton or electrical stimulation devices for patients with partial paralysis, in which sensors are implanted in a location that contains muscles the user can still control. The system could then reliably deliver control signals to the exoskeleton or stimulation device to control movement.

Another set of embodiments may apply to the consumer domain, where the system can be used to control a variety of consumer electronics (e.g. intention detection). For example, an embodiment can be utilized as a video game controller, as an avatar controller for virtual/augmented reality, as a keyboard or mouse, or as a supplemental control signal for autonomously driving cars. New consumer applications can be built, e.g., by third-parties, via an exposed application programming interface (API).

Some embodiments further include a non-transitory computer-readable storage medium storing program code including instructions that, when executed by a processor or processors, cause the one or more processors to perform one of the methods of calibrating or training or using a brain-machine interface (BMI) system, as described herein. Non-exclusive examples of non-transitory computer-readable storage media include any medium that can store program code, such as a USB flash drive, a removable hard disk, a read-only memory (ROM), a random access memory (RAM), a magnetic disk, or an optical disc.

Experimental Results

Two embodiments of a BMI system were successfully tested in rat and human subjects. The first embodiment included an intramuscular electrode array and was tested in rats. The second embodiment, instead, included a matrix of high-density surface EMG electrodes and was tested in healthy human subjects. The following sections provide an overview of the performed experiments demonstrating the feasibility and potential of the BMI embodiments.

Intramuscular Implementation of a BMI in Free-Behaving Rats

A series of experiments was conducted in rats to evaluate the potential of an intramuscular implementation of a BMI embodiment. 16-channel micro-wire electrode arrays were used to chronically record neuromuscular signals from a targeted muscle and an operant conditioning paradigm to assess rats' ability to learn to control different motor units belonging to the same muscle independently.

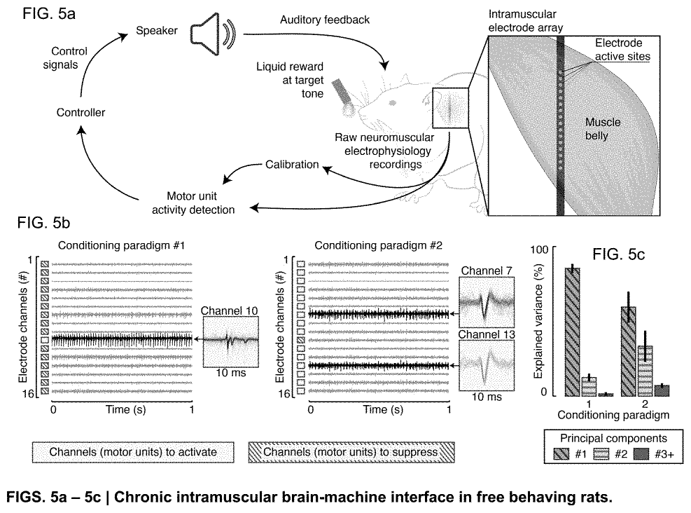

Using a motor unit detection model, the activity of the sampled motor units was linked to an auditory signal that was fed back in real-time to the rat (FIG. 5a). By rewarding rats when producing a target motor unit activity (associated with a specific auditory tone), it was evaluated whether two different patterns of motor unit activity could be enforced. First, it was assessed whether rats could learn to selectively activate a small subset of motor units among those sampled by the implanted electrode. For this, the measured motor units were clustered in two ensembles. The first ensemble (E1) included the set of motor units with the lowest recruitment threshold recorded from a single channel, while the second ensemble (E2) included all the remaining units. The auditory feedback signal was controlled with a differential transform, rewarding a stronger activity in E1 units compared with E2 units. A gain was used to adjust the task difficulty over time with the objective to promote an increasingly selective activation of E1 units. After a week of training with this paradigm, the two ensembles were switched, and an evaluation was made as to whether the rats could learn to selectively activate the motor units in E2.

Preliminary results indicated that rats can successfully learn to selectively activate each of the two motor unit ensembles (see FIG. 5b for representative recordings in one rat). In addition, it was found that the amount of variance explained by the first principal component of the firing rate of the recorded motor units tended to decrease as a selective activation of E2 and suppression of E1 was enforced (FIG. 5c). This suggests that the operant conditioning paradigm successfully increased the dimensionality of the excitatory drive sent to the targeted muscle, highlighting the key advantage of the BMI embodiment compared to existing systems. Indeed, while existing systems (which aim to detect motor commands from the natural motor repertoire) have an intrinsically fixed number of control signals that can be detected from a group of recorded muscles (dictated by the number of functions they carry out), the BMI embodiment exploits the brain ability to learn new skills to expand with neurofeedback training the number of control signals that can be extracted from each muscle.

Non-Invasive Implementation of the Proposed BMI in Healthy Human Subjects

A grid of high-density surface EMG electrodes was used to evaluate the potential of non-invasive implementations of a BMI embodiment in healthy human subjects. In particular, a 64-channel grid of electrodes was used to detect motor unit activity form the biceps muscle and an evaluation made as to the subjects' ability to learn to control different motor units independently to operate a computer mouse. To minimize potential confounds caused by the high susceptibility of surface EMG recordings to motion artefacts, elbow flexion-and-extension and wrist pronation-and-supination movements were constrained by a sensorized orthosis effectively only allowing subjects to perform isometric biceps contractions.

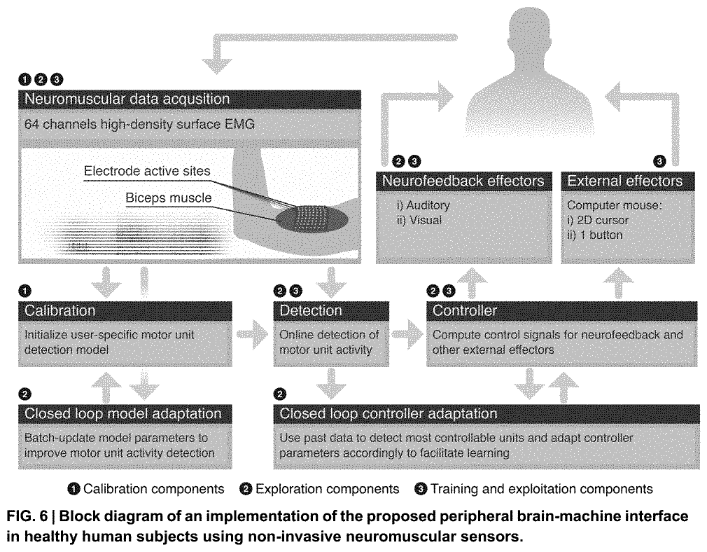

Experiments where divided in three phases: (i) calibration, (ii) exploration, and (iii) training-and-exploitation (See FIG. 6 for an overview of each phase). During calibration, subjects were instructed to perform a series of weak biceps contractions aimed at initializing the motor unit detection model used for online motor unit activity detection. In the exploration phase, subjects were provided with auditory and visual neurofeedback signals linked to their motor unit activity. During this phase, subjects were encouraged to use these neurofeedback signals to attempt to activate different motor units independently, while closed loop adaptation algorithms were used to fine tune the motor unit detection model and the controller module until at least three controllable motor units are found. Finally, during the training-and-exploitation phase, the three most controllable motor units were used to operate a computer cursor and perform a center-out task. Auditory and visual neurofeedback signals remained active.

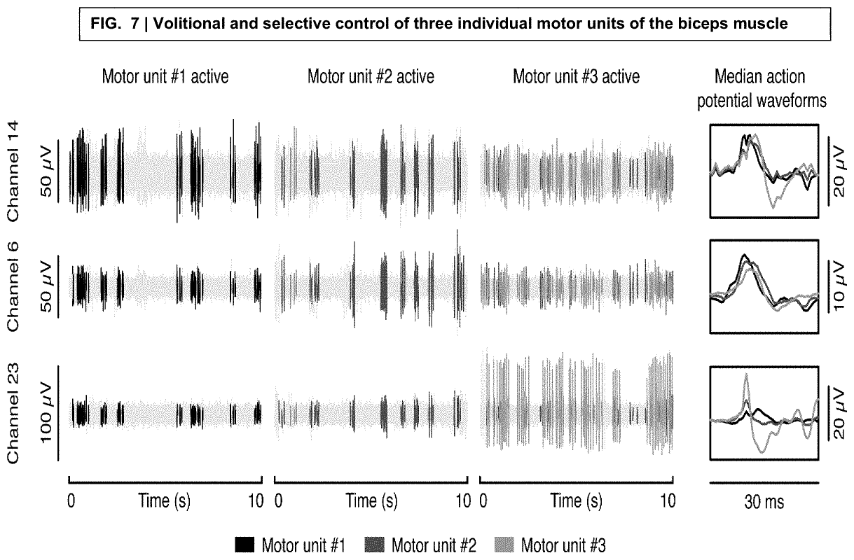

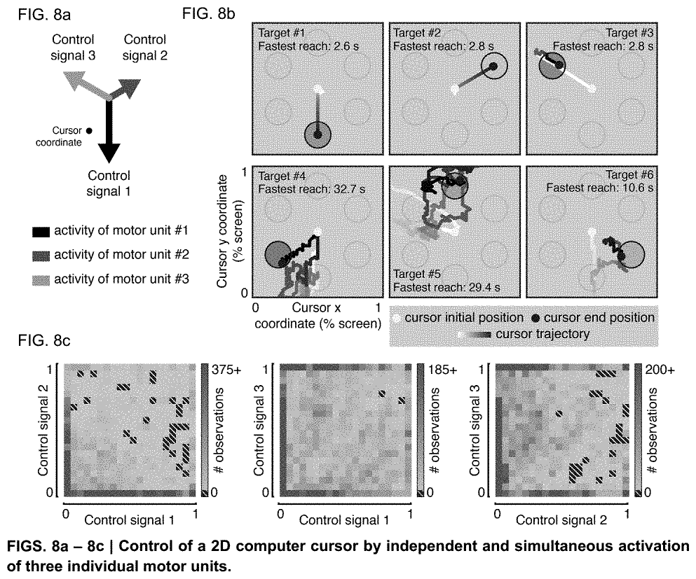

It was found that subjects were able to successfully integrate the provided neurofeedback signals and learn to activate multiple motor units independently. FIG. 7 reports segments of measured neuromuscular activity where a subject was able to selectively activate three individual motor units. Using these units, the same subject was able to control a 2D computer cursor and perform a center-out task (FIG. 8a-8c). In particular, the subject successfully managed to reach both targets requiring a selective activation of each single motor unit (FIG. 8b, top row) and targets requiring simultaneous activations of different motor unit pairs (FIG. 8b, bottom row). Task performance, however, was largely superior in the former case, as indicated by the fastest reach times for each target. Finally, it was found that the control signals generated throughout the center-out task (of 20 minutes duration), were spanning the whole control space, further indicating that the subject was able to independently control all three motor units.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the embodiments (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed embodiments and does not pose a limitation on the scope of the disclosure unless otherwise claimed. No language in the specification should be construed as indicating any non-claimed element as essential to the practice of the embodiments.

Exemplary embodiments are described herein. Variations of those exemplary embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, the scope of the disclosure includes all modifications and equivalents of the subject matter recited herein and in the claims appended hereto as permitted by applicable law. Moreover, any combination of the above-described elements in all possible variations thereof is encompassed by the disclosure unless otherwise indicated herein or otherwise clearly contradicted by context.

1. A brain-machine interface (BMI) system, comprising:
one or more implantable or non-implantable sensors, each of the one or more sensors configured to detect or measure electrophysiological activity of motor units and to transmit an electrophysiological activity signal;
one or more wearable apparatuses configured to be worn by or attached to a user and configured to receive and process the one or more electrophysiological activity signals transmitted by the one or more sensors, and configured to transmit the processed signals to one or more processing units;
the one or more processing units, configured to receive the processed signals from the one or more wearable apparatuses and to produce control signals based on the received processed signals using one or more statistical models and/or trained machine learning algorithms; and
one or more effectors configured to receive the control signals and configured to transduce the control signals into a haptic, tactile, chemical, mechanical, auditory, visual, and/or electrical stimuli so as to provide feedback to the user and/or to control operation of an external effector.

2. The BMI system of claim 1, wherein the one or more wearable apparatuses process the one or more electrophysiological activity signals by applying one or more of a filtering algorithm, a down-sampling algorithm, a signal detection algorithm to the one or more electrophysiological activity signals.

3. The BMI system of claim 1, wherein at least one of the one or more implantable sensors includes one or multiple electrodes and an RF transceiver.

4. The BMI system of claim 1, wherein at least one of the one or more sensors is non-invasive and positioned on the skin near targeted nerves or muscles.

5. The BMI system of claim 1, wherein at least one of the one or more sensors includes a non-invasive high-density grid of surface EMG electrodes.

6. The BMI of claim 5, wherein the high-density grid includes a grid of electrodes with a minimum of 16 electrodes and a maximum inter-electrode distance of 10 mm.

7. The BMI system of claim 3, wherein the one or multiple electrodes are configured to be implanted intradermally, intramuscularly or on the epimysium of a targeted muscle.

8. The BMI system of claim 3, wherein the one or multiple electrodes are configured to be implanted on the epineurium or within the nerve innervating a targeted muscle.

9. The BMI system of claim 1, wherein the one or more effectors include at least one neurofeedback effector.

10. The BMI system of claim 1, wherein the one or more effectors include at least one external effector.

11. The BMI system of claim 10, wherein the at least one external effector comprises one of a computing device, a mechanical actuator, a mechanical transducer, an exoskeleton, a robotic manipulandum, a prosthesis, or a smart phone.

12. A non-transitory computer-readable medium storing instructions, which when executed by one or more processors cause the one or more processors to:
receive one or more processed signals from one or more wearable apparatuses, each of the one or more processed signals representing measured electrophysiological activity of a motor unit of a user;
produce control signals based on the received processed signals using one or more statistical models and/or trained machine learning algorithms; and
transmit the control signals to one or more effectors configured to transduce the control signals into a haptic, tactile, chemical, mechanical, auditory, visual, and/or electrical stimuli so as to provide feedback to the user and/or to control operation of an external effector.

13. The non-transitory computer-readable medium of claim 12, wherein the one or more effectors include at least one external effector, and wherein the at least one external effector comprises one of a computing device, an exoskeleton, a prosthesis, or a smart phone.

14. The non-transitory computer-readable medium of claim 12, wherein the one or more wearable apparatuses process the one or more electrophysiological activity signals by applying one or more of a filtering algorithm, a down-sampling algorithm, a signal detection algorithm to the one or more electrophysiological activity signals.

15. The non-transitory computer-readable medium of claim 12, wherein the one or more effectors include at least one neurofeedback effector.

16. The non-transitory computer-readable medium of claim 12, wherein the one or more effectors include at least one external effector and wherein the at least one external effector comprises one of a mechanical actuator, a mechanical transducer, and a robotic manipulandum,
